(19) United States
(12) Patent Application Publication
(10) Pub. No.: US 2006/0141603 A1
(43) Pub. Date: Jun. 29, 2006

(54) Methods and compositions for selectively enriching microbes

(76) Inventor: Michael Grant, Bainbridge Island, WA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/539,765

(22) PCT Filed: Dec. 19, 2003
(86) PCT No.: PCT/US03/40806

§ 371 Date: Feb. 8, 2006

(51) Int. Cl. C12P 21/06; C12N 1/20

(52) U.S. Cl. 435/252.8; 435/69.1

(57) ABSTRACT

The present invention provides methods and materials to selectively enrich target microbes in a sample.

This application claims priority from U.S. application Ser. No. 60/435,639 filed Dec. 20, 2002.

### GOVERNMENT FUNDING

The invention described herein was developed with support from the Food and Drug Administration. The U.S. Government has certain rights in the invention.

### FIELD OF THE INVENTION

The invention relates generally to methods for enriching the numbers of target microbes in test samples that may contain a mixture of microbial species. More specifically, the invention relates to a selection procedure involving incubating a sample that may contain competitor microbes and target microbes under acidic conditions such that the competitor microbes are growth inhibited or killed, but where the target microbes survive. The surviving target microbes can then be detected using available procedures.

### BACKGROUND OF THE INVENTION

Enterohemorrhagic Escherichia coli has emerged in recent years as the predominant cause of hemorrhagic colitis in humans. It may be present in foods of animal-origin or plant-origin, as well as in water, and has been implicated as a cause of food poisoning. Diarrhea, hemorrhagic colitis, hemolytic uremic syndrome, and thrombocytopenic purpura are the principal manifestations of illness.

The pathogenicity of enterohemorrhagic E. coli appears to be associated with a number of virulence factors, including the production of several cytotoxins. These toxins are collectively referred to as verotoxins or Shiga-like toxins because the Shiga-like toxin of E. coli closely resembles the Shiga toxin of Shigella dysenteriae type 1. Although more than sixty E. coli serotypes produce Shiga-like toxins and more are being identified as capable of producing Shiga-like toxins, serotype O157:H7 is the predominant pathogen in the enterohemorrhagic E. coli group and the one associated most frequently with human infections worldwide.

The initial symptoms of hemorrhagic colitis generally occur between one and two days after eating contaminated food, although longer periods (3-5 days) have been reported. Symptoms start with mild, non-bloody diarrhea that may be followed by a period of crampy abdominal pain and short-lived fever. The initial diarrhea increases in intensity during the next 24-48 hours to a 4 to 10-day phase of overtly bloody diarrhea accompanied by severe abdominal pain and moderate dehydration.

Several life-threatening complications may occur in hemorrhagic colitis patients; hemolytic uremic syndrome is the most common. The onset of hemolytic uremic syndrome is approximately a week after the onset of gastrointestinal symptoms. Characteristic symptoms are pallor, intravascular destruction of red blood cells (microangiopathic hemolytic anemia), depressed platelet counts (thrombocytopenic), lack of urine formation (oligo-anuria), swelling (edema), and acute renal failure. Hemolytic uremic syndrome occurs most often in children under the age of ten years. Approximately half of hemolytic uremic syndrome patients require dialysis, and the mortality rate is 3-5%.

Other hemolytic uremic syndrome-associated complications may include seizures, coma, stroke, colonic perforation, pancreatitis, and hypertension. Approximately 15% of cases lead to early development of chronic kidney failure. Insulin-dependent diabetes may also persist in hemolytic uremic syndrome patients. A small number of hemolytic uremic syndrome cases may recur.

A second complication associated with E. coli O157:H7 is thrombotic thrombocytopenic purpura. This condition resembles HUS except that it generally causes less renal damage; has significant neurological involvement, e.g., central nervous system deterioration, seizures, and strokes; and is restricted primarily to adults.

Enterohemorrhagic Escherichia coli presents a significant health hazard. Accordingly, improved methods to determine if clinical, food, water, or environmental samples contain Enterohemorrhagic bacteria are necessary to ensure the proper diagnosis and treatment of patients, as well as ensuring the safety of food and waters.

### SUMMARY OF THE INVENTION

The present invention provides methods and kits that can be used to produce a test sample in which a target microbe is enriched.

The methods used to enrich a target microbe in a test sample involve incubating a first sample that may contain competitive microbes and a target microbe under acidic conditions that inhibit or kill competitor microbes in the first sample and thereby provide a growth advantage to the target microbe. The surviving target microbes can be detected immediately or subjected to conditions permitting additional growth to facilitate detection.

In one embodiment, the target microbe is a bacterium. In some embodiments, the target microbe is selected from Escherichia, Salmonella, Staphylococcus, Klebsiella, Listeria, Morganella, Enterobacter, Serratia, Yersinia, Bacillus, Shigella and Hafnia. For example, the methods of the invention can be used with samples suspected of containing target microbes such as pathogenic and non-pathogenic Escherichia coli. In some embodiments, the target microbe is Shigella. In other embodiments, the target microbe is a pathogenic bacterium. For example, the methods of the invention can be used for enrichment of pathogenic Escherichia coli such as enterohemorrhagic Escherichia coli, enterotoxigenic Escherichia coli (ETEC), enteropathogenic Escherichia coli (EPEC) and the like. In further embodiments, the target microbe is the O157:H7 strain of Escherichia coli.

In one embodiment, the acidic condition includes an acidic medium that inhibits the growth of competitor microbes when they are later incubated in growth media. In some embodiments, the pH of the acidic medium is between 1 and 6. In other embodiments, the pH of the acid medium is between 1 and 4. In further embodiments, the pH of the acidic medium is between 1 and 2. In still further embodiments, the pH of the acidic medium is between 2 and 4. In a preferred embodiment, the pH of the acidic medium is between 1.5 and 2.5.

In another embodiment, the acid condition includes an acidic medium that causes death of the competitor microbes during incubation in the acidic medium. In some embodiments, the pH of the acidic medium is between 1 and 6. In other embodiments, the pH of the acid medium is between 1 and 4. In further embodiments, the pH of the acidic medium is between 1 and 2. In still further embodiments, the pH of the acidic medium is between 2 and 4. In a preferred embodiment, the pH of the acidic medium is between 1.5 and 2.5.

In one embodiment, the first sample is incubated in an acidic medium that inhibits the growth of the competitive microbes when they are later incubated in a growth medium. In some embodiments, the first sample is incubated in the acidic medium for a time period between 0.5 and 10 hours. In other embodiments, the first sample is incubated in the acidic medium for a time period between 1 and 6 hours. In further embodiments, the first sample is incubated in the acidic medium for a time period between 1 and 4 hours. In still further embodiments, the first sample is incubated in the acidic medium for a time period between 1 and 2 hours. In many embodiments, the first sample is incubated in the acidic medium for a time period between 1.5 and 3 hours.

In another embodiment, the microbes are incubated in an acid medium for a period of time that causes death of the competitive microbes. In some embodiments, the first sample is incubated in the acidic medium for a time period between 0.5 and 10 hours. In other embodiments, the first sample is incubated in the acidic medium for a time period between 1 and 6 hours. In further embodiments, the first sample is incubated in the acidic medium for a time period between 1 and 4 hours. In still further embodiments, the first sample is incubated in the acidic medium for a time period between 1 and 2 hours. In many embodiments, the first sample is incubated in the acidic medium for a time period between 1.5 and 3 hours.

In another embodiment the first sample is incubated in an acidic medium that inhibits the growth of the competitive microbes when they are later incubated in a growth medium. In some embodiments, the temperature is within the range of about 45° C. to about 70° C. In other embodiments, the temperature is within the range of about 35° C. to about 45° C. In further embodiments, the temperature is within the range of about 25° C. to about 35° C. In still further preferred embodiments, the temperature is within the range of about 5° C. to about 20° C. In many embodiments, the temperature is within the range of about 20° C. to about 25° C.

In another embodiment the microbes are incubated in an acidic medium at a temperature that causes death of the competitive microbes. In some embodiments, the temperature is within the range of about 45° C. to about 70° C. In other embodiments, the temperature is within the range of about 35° C. to about 45° C. In further embodiments, the temperature is within the range of about 25° C. to about 35° C. In still further preferred embodiments, the temperature is within the range of about 5° C. to about 20° C. In many embodiments, the temperature is within the range of about 20° C. to about 25° C.

In another embodiment, the microbes are incubated in an acidic medium at a temperature, pH, and for a length of time that inhibits the growth of the competitive microbes when they are later incubated in a growth medium. In one embodiment, the temperature is within the range of about 5° C. to about 35° C., the time is within a range of about 1 hour to about 3 hours, and the pH is between about 1.5 and about 3. In another embodiment, the temperature is within the range of about 20° C. to about 25° C., the time is within a range of about 1 hour to about 3 hours, and the pH is between about 1.5 and about 2.5. In further embodiments, the temperature is about 22° C., the time about 2 hours, and the pH is about 2.0.

In another embodiment, the microbes are incubated in an acidic medium at a temperature, pH, and for a length of time that causes death of the competitive microbes when they are later incubated in a growth medium. In one embodiment, the temperature is within the range of about 5° C. to about 35° C., the time is within a range of about 1 hour to about 3 hours, and the pH is between about 1.5 and about 3. In another embodiment, the temperature is within the range of about 20° C. to about 25° C., the time is within a range of about 1 hour to about 3 hours, and the pH is between about 1.5 and about 2.5. In further embodiments, the temperature is about 22° C., the time about 2 hours, and the pH is about 2.0.

In one embodiment, the acidic medium includes a selective agent. In some embodiments, the selective agent is a phage or virus that infects the competitive microbes. In further embodiments, the selective agent is an antibiotic. In still further embodiments, the selective agent is more than one antibiotic. In other embodiments, the selective agent is a nutritional supplement. Examples of non-antibiotic selective agents include organic or inorganic chemicals such as tellurite, selenite, sorbitol, and the like. In another embodiment the acidic medium does not include a selective agent.

In one embodiment, the growth medium used after selection with the acidic medium includes a selective agent. In some embodiments, the selective agent is a phage or virus that infects the competitive microbes. In further embodiments, the selective agent is an antibiotic. In still further embodiments, the selective agent is more than one antibiotic. In other embodiments, the selective agent is a nutritional supplement. Examples of non-antibiotic selective agents include organic or inorganic chemicals such as tellurite, selenite, sorbitol, and the like. In another embodiment, the growth medium does not include a selective agent.

In another embodiment the acidic medium and the growth medium each include a selective agent. In some embodiments, the selective agent is a phage or virus that infects the competitive microbes. In further embodiments, the selective agent is an antibiotic. In still further embodiments, the selective agent is more than one antibiotic. In other embodiments, the selective agent is a nutritional supplement. Examples of non-antibiotic selective agents include organic or inorganic chemicals such as tellurite, selenite, sorbitol, and the like.

In another embodiment the acidic medium includes a selective agent and the growth medium does not include a selective agent. In some embodiments, the selective agent is a phage or virus that infects the competitive microbes. In further embodiments, the selective agent is an antibiotic. In still further embodiments, the selective agent is more than one antibiotic. In other embodiments, the selective agent is a nutritional supplement. Examples of non-antibiotic selective agents include organic or inorganic chemicals such as tellurite, selenite, sorbitol, and the like.

In another embodiment the acidic medium does not include a selective agent and the growth medium includes a selective agent. In some embodiments, the selective agent is a phage or virus that infects the competitive microbes. In further embodiments, the selective agent is an antibiotic. In still further embodiments, the selective agent is more than one antibiotic. In other embodiments, the selective agent is a nutritional supplement. Examples of non-antibiotic selective agents include organic or inorganic chemicals such as tellurite, selenite, sorbitol, and the like.

Kits are also provided by the invention.

In one embodiment, the invention provides a kit that includes packaging material and an acidic medium. The acidic medium can be a dry medium. In other embodiments, the acidic medium is in liquid form. In some embodiments, the acidic medium has, or upon re-hydration will have, an acidic pH.

In another embodiment, the invention provides a kit that includes packaging material, acidic medium, and growth medium. The acidic medium and the growth medium can be in dry form. In other embodiments, the acidic medium and the growth medium are in liquid form. In some embodiments, the acidic medium has, or upon re-hydration will have, an acidic pH.

In another embodiment, the invention provides a kit that includes packaging material, media, and a pH modifier that can be added to the media to make the media into an acidic medium. The media can be in dry form. In other embodiments, the media is in liquid form.

The pH modifier can be an acidic buffer. Alternatively, the pH modifier can be an organic acid. In many embodiments the pH modifier is an inorganic acid.

In another embodiment, the invention provides a kit that includes packaging material, media, a first pH modifier that causes the media to become an acidic medium, and a second pH modifier that causes the acidic medium to become growth medium. The media can be in dry or liquid form. The first pH modifier can be an acidic buffer, an organic acid or an inorganic acid. The second pH modifier can be a basic buffer, an organic base or an inorganic base.

In another embodiment, the invention provides a kit that includes an acidic medium, a growth medium, and a means for detecting microbes. The means for detecting microbes can be used to detect bacteria. In some embodiments, the means for detecting microbes can be used to detect pathogenic bacteria. In other embodiments, the means for detecting microbes can be used to detect Shigella. In further embodiments, the means for detecting microbes can be used to detect Escherichia coli. In further embodiments, the means for detecting microbes can be used to detect pathogenic Escherichia coli such as enterohemorrhagic Escherichia coli, enterotoxigenic Escherichia coli (ETEC), enteropathogenic Escherichia coli (EPEC) and the like. In still further embodiments, the means for detecting microbes can be used to detect the O157:H7 strain of Escherichia coli.

### DESCRIPTION OF THE DRAWINGS

FIG. 1 shows the comparative growth of ten different cultures of pathogenic enterohemorrhagic E. coli in the standard enrichment procedure (E) versus the enrichment procedure described in Example I (T). The horizontal axis indicates the ten different pathogenic bacterial stains used in the assay, and the vertical axis indicates cell density (×10⁸ per milliliter of medium).

FIG. 2 shows the comparative growth of ten different cultures of pathogenic enterohemorrhagic E. coli in the standard enrichment procedure (E) versus the enrichment procedure described in Example II after the cultures were aged for 44 days (T). The horizontal axis indicates the ten different pathogenic bacterial stains used in the assay, and the vertical axis indicates cell density (×10⁸ per milliliter of medium).

FIG. 3 provides a photograph of an agarose gel showing that PCR can be used to facilitate detection of acid enriched microbial samples but use of FDA approved enrichment procedures interferes with PCR detection. Lanes 1 and 2 show that bands typical of the gad gene were generated using two versions of the inventive acid enrichment method. Lane 4 shows no band was generated when enrichment was performed according to FDA accepted procedures. Lanes 3 and 5 are positive controls showing the gad fragment. Lanes 6 and 7 show bands typical of the st×1 and st×2 fragments that were generated by PCR amplification of template nucleic acids obtained by the inventive acid enrichment method. Lane 9 shows that no st×1 or 2 band was generated when FDA enrichment was utilized. Lanes 8 and 10 were positive controls showing the st×1 and st×2 fragments.

FIG. 4 graphically illustrates that greater numbers of E. coli O157:H7 cells are recovered by the acid enrichment procedures of the invention than by enrichment procedures commonly used by the Federal Drug Administration (FDA), United States Department of Agriculture (USDA) and Canadian regulatory agencies. The acid enrichment procedure of the invention was also superior to enrichment procedures that employ Buffered Peptone Water (BPW). After enrichment by the indicated procedure, E. coli O157:H7 cells were identified by growth on TCSMAC agar. E. coli O157:H7 cells produce sorbitol negative colonies on TCSMAC.

FIG. 5 graphically illustrates that greater numbers of E. coli O157:H7 cells are recovered by the acid enrichment procedures of the invention than by enrichment procedures commonly used by the Federal Drug Administration (FDA), United States Department of Agriculture (USDA) and Canadian regulatory agencies. The acid enrichment procedure of the invention was also superior to enrichment procedures that employ Buffered Peptone Water (BPW). After enrichment by the indicated procedure, E. coli O157:H7 cells were identified by growth on Rainbow agar. E. coli O157:H7 cells produce black colonies on Rainbow agar.

FIG. 6 graphically illustrates that greater numbers of E. coli O157:H7 cells are recovered by the acid enrichment procedures of the invention than by enrichment procedures commonly used by the Federal Drug Administration (FDA), United States Department of Agriculture (USDA) and Canadian regulatory agencies. The acid enrichment procedure of the invention was also superior to enrichment procedures that employ Buffered Peptone Water (BPW). After enrichment by the indicated procedure, E. coli O157:H7 cells were identified by growth on Rainbow agar with tellurite and novobiocin. E. coli O157:H7 cells produce black colonies on Rainbow agar plus tellurite and novobiocin.

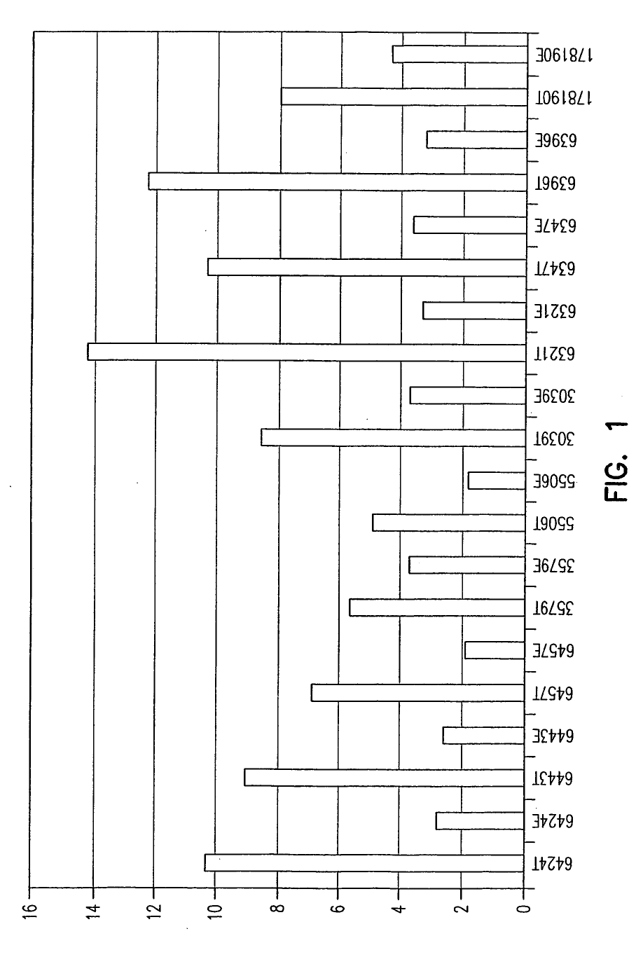

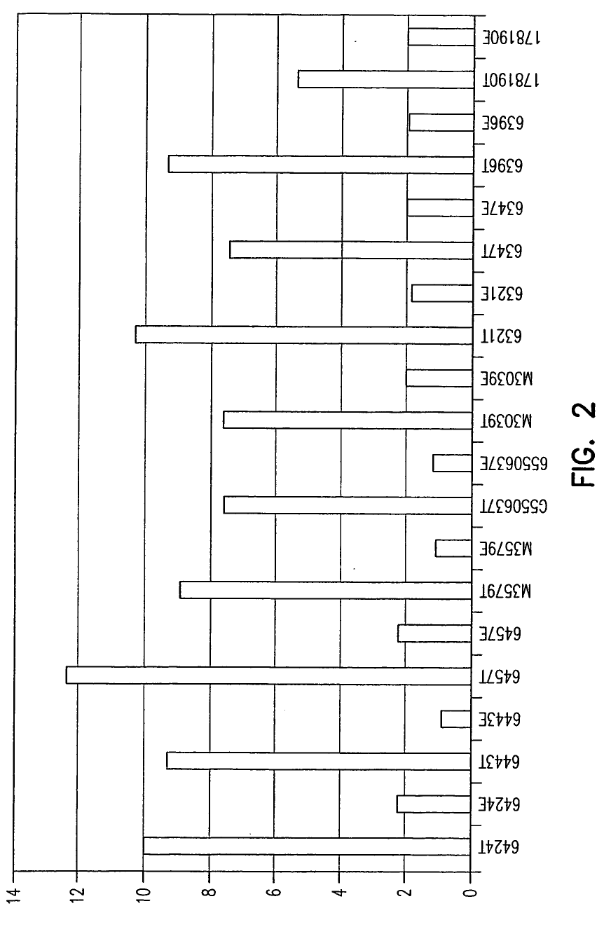

### DETAILED DESCRIPTION OF THE INVENTION

Methods to detect microbes, such as pathogenic bacteria, play an important role in everyday life. Such methods are used to monitor food and water supplies to ensure the safety of food and community drinking water. These methods are also used in clinics for diagnosis and to monitor treatment of infectious diseases. Improved methods to detect microbes that decrease the time necessary to obtain results, or increase the accuracy of the results obtained, can save the lives of persons infected with such microbes. Improved detection methods also allow communities to respond more rapidly to potential outbreaks of microbes that may be pathogenic.

Methods used to detect microbes in a sample generally include an enrichment step, where the number of microbes in a sample is increased, and a detection step, where a target microbe is actually detected. The enrichment step involves increasing the number of target microbes being tested for in a sample. The enrichment step may also decrease the number of competing microbes in the sample that are not target microbes. Decreasing the number of competing microbes and increasing the number of target microbes in a sample allows for more accurate detection of target microbes in a sample because the background from the competing microbes is reduced. In addition, increasing the number of target microbes during the enrichment step may allow the target microbes to reach a detectable level.

The standard assay used by the Food and Drug Administration to detect enterohemorrhagic E. coli uses an enrichment medium that contains components to allow growth of all heterotrophic bacteria, plus a collection of three antibiotics and bile salts to inhibit competing microbes.

The present invention provides a method to enrich microbes present in a sample, generally allowing more rapid detection and analysis of such microbes. These microbes include pathogenic bacteria that may present a health hazard to humans and animals exposed to the pathogenic bacteria.

Generally, the invention involves incubating a first sample under acidic conditions to produce a second sample, and then incubating the second sample in a growth medium to produce a test sample. The acidic medium is selected to allow the target microbe to multiply when incubated in the growth medium, and to cause some or all of the competing microbes to be killed or inhibited from growing when they are incubated in growth medium. Thus, this method allows the number of competing microbes to be reduced and the number of target microbes to be enriched in a test sample. This test sample is then used in the detection step to determine if the starting sample contained a target microbe, such as a pathogenic bacterium.

An unexpected discovery of the inventive method is that competitive or non-pathogenic microbes may be selected against by incubation in an acidic medium in the absence of additional selective agents, such as antibiotics. Also, target microbes such as pathogenic bacteria, may be positively selected by incubation in an acidic medium. This feature of the inventive method allows the target bacteria to be enriched by incubation in the growth medium in the absence of commonly used antibiotics or other selective agents. In some instances, use of the inventive method allows a target microbe to replicate at a more rapid rate than previously employed enrichment methods because the target microbe is not required to overcome negative selection resulting from the presence of a selective agent. Moreover, the present methods allow faster detection of the enriched microbes because they do not interfere with detection by polymerase chain reaction (PCR), ELISA or other rapid detection methods.

In addition, certain selective agents have been shown to affect the results of methods used to detect the presence of a target microbe in a sample. Johnson et al., Appl. Envir. Microbiol., 61:386 (1995). For example, experiments described herein illustrate that procedures generally accepted by the FDA that rely upon use of several selective agents actually interfere with PCR analysis of pathogen enriched cultures (see Example VI). Accordingly, the present invention can offer many advantages over currently available methods for microbe enrichment that include, increased growth rate of a target microbe due to the absence of a selective agent, reduced interference from a selective agent during detection of a target microbe, increased ease of use due to the absence of a selective agent, and decreased expense due to the absence of a selective agent. Alternatively, selective agents may be included in the acidic medium or the growth medium to provide additional selection in addition to acidic pH.

Definitions

Abbreviations: enterohemorrhagic E. coli (EHEC), enteropathogenic E. coli (EPEC), enterotoxigenic E. coli (ETEC), enzyme linked immunosorbant assay (ELISA), Escherichia coli (E. coli), polymerase chain reaction (PCR), tryptone soy broth (TSB).

The term “acid media” or “acidic medium” refers to a medium or media having a pH less than 7.0.

The term “antibiotic” refers to a chemical that kills or inhibits the growth of a microbe that is not resistant to the antibiotic. Examples of antibiotics include, but are not limited to, ampicillin, streptomycin, rifampicin, tetracycline, and the like. Numerous antibiotics are known in the art. Merck Index, 13^{th} edition, Merck & Co., Whitehouse Station, N.J. 2001.

The term “basic media” or “basic medium” refers to a medium or media having a pH greater than 7.0.

The term “bodily sample” refers to a sample obtained from an organism. Examples of bodily samples include, but are not limited to, blood, urine, saliva, tears, skin, mucus, bodily secretions, and bodily swabs.

The term “competitor microbe” refers to microbe that is selected against in a sample through incubation in an acidic medium followed by incubation in a growth medium. In one example, the competitor microbe may be a non-enterohemorrhagic E. coli contained within a sample that is being tested for a target enterohemorrhagic E. coli.

The term “environmental sample” refers generally to a specimen that is to be tested to determine whether the specimen contains a target microbe. Examples of environmental samples include water, food, meat, swabs from instruments or rooms being tested for microbial contamination, soil, fecal matter, cattle feed, plants, animal products, and the like.

The term “growth inhibit” or “growth inhibited” describes a characteristic of a competitor microbe that is induced by incubating the competitor microbe in an acidic medium, and that causes growth of the competitor microbe to be eliminated or decreased during later incubation in a growth medium.

The term “growth media” or “growth medium” refers to a medium or media that is selected to promote replication of a microbe. Such a medium may be selected based in the needs of a particular target microbe. Many types of media are well known in the art that allow survival and replication of microbes. Sambrook et al., Molecular Cloning: A Laboratory Manual, 3rd edition, Cold Spring Harbor Press, Cold Spring Harbor, N.Y. (2001).

The term “media” or “medium” refers to a support for microbial growth. Examples of media include, but are not limited to, nutrient broths, agars, and powdered components that can be used to form nutrient broths and agars. Media may be in liquid form, semi-solid form, or in dry form. Many types of media are known in the art and include, but are not limited to, GYT medium, LB medium, M9 minimal medium, NZCYM medium, NZYM medium, SOB medium, SOC medium, TB medium, TSB medium, 2×YT medium, and agar plates. Numerous media recipes are known and have been described. Sambrook et al., Molecular Cloning: A Laboratory Manual, 3rd edition, Cold Spring Harbor Press, Cold Spring Harbor, N.Y. (2001).

The term “microbe” refers to a biological entity that is able to replicate in a medium. Examples of microbes include, but are not limited to, bacteria, mold, fungus, and nematodes. The present invention may be used to selectively enrich a sample for numerous types of target microbes in a sample by incubating the sample in an acidic medium and then in a growth medium to enrich the target microbe in the sample. The conditions required to determine the specific conditions to achieve enrichment of a specific target microbe can readily be determined by one of skill in the art.

The term “negative selective agent” refers to a chemical or biological agent that inhibits the growth of one microbe over another.

The term “neutral media” or “neutral medium” refers to a medium or media having pH 7.0.

The term “non-inhibitory” refers to a medium in which the growth of a microbe is not inhibited because the medium lacks a selective agent. In some embodiments, a non-inhibitory medium is a medium in which growth of a microbe is not inhibited at a selected pH. The pH of such a non-inhibitory medium may be adjusted to conform to the preferences of a microbe that is grown in the medium.

The term “pH modifying agent” includes a compound that causes an increase or decrease in the pH of a medium. For example, a pH modifying agent can be an organic acid or base. A pH modifying agent can also be an inorganic acid or base. Alternatively, a pH modifying agent can be a buffer that acts to increase or decrease the pH of a medium to which it is added. Examples of such buffers include, but are not limited to, acetete buffer, bicine buffer, borate buffer, citrate buffer, phosphate buffer, cysteine buffer, glycine buffer, HEPES buffer, maleate buffer, carbonate buffer, diethanolamine buffer, sulfate buffer, sulfite buffer and the like. Such buffers are well known in the art and are described. CRC Handbook of Chemistry and Physics, 83^{rd} edition, 7-13-7-15, CRC Press (2002); Harlow et al., Antibodies: A Laboratory Manual, page 319 (Cold Spring Harbor Pub. 1988). Examples of acids include, but are not limited to, sulfuric, nitric, hydrochloric, formic, phosphoric, and acetic. Examples of bases include, but are not limited to, ammonia, sodium hydroxide, and potassium hydroxide.

The term “positive selective agent” refers to a chemical or biological agent that promotes the growth of one microbe over another.

The term “selective agent” as used herein refers to a chemical or biological agent other than pH that selectively alters the growth of one microbe over another. Examples of selective agents include, but are not limited to, antibiotics, phages, viruses, and nutrients.

The terms “selective enrichment” or “selectively enrich” refer to the process of causing the relative population of a select microbe in a sample to increase relative to the population of competitor (non-target) microbes in the sample. For example, a low relative population of a target microbe may be initially present in a sample when compared to other microbes present in the sample. A target microbe in the sample may be selectively enriched by incubating the sample under conditions in which a competitor (non-target) microbe in the sample is killed or growth inhibited, and then be incubated under conditions where the target microbe multiplies at a higher rate than a competitor (non-target) microbe in the sample. This process will selectively enrich the sample for a target microbe by increasing the relative population of the target microbe relative to the competitor microbe.

The term “target microbe” refers to a microbe that is selectively enriched in a sample through incubation in an acidic medium followed by incubation in a growth medium. In some embodiments, the target microbe may be an enterohemorrhagic E. coli (EHEC), enteropathogenic E. coli (EPEC), or enterotoxigenic E. coli (ETEC). Such pathogens may be contained within a sample that contains other bacteria such as other pathogenic or non-pathogenic bacteria.

Incubation in an Acidic Medium

The methods of the present invention involve incubating a first sample containing a competitor microbe and a target microbe in an acidic medium to produce an enriched sample. 25 The methods of the invention can also include, incubating the second sample in a growth medium to produce a test sample that is enriched for the target microbe. It is understood to those of skill in the art that the inventive method may be used to enrich for numerous types of microbes and that enterohemorrhagic E. coli are used as an example to illustrate the invention, but not to limit the invention. For example, the methods and kits of the invention can be used to enrich aerobic and anaerobic microbes, such as bacteria.

The acidic medium may be a microbial medium that can be acidified to provide a selective growth advantage to a target microbe as compared to a competitor microbe. Numerous examples of media exist that may be acidified and used within the inventive method. Examples of such media for bacterial enrichment include, but are not limited to, GYT medium, LB medium, M9 minimal medium, NZCYM medium, NZYM medium, SOB medium, SOC medium, TB medium, 2×YT medium, BHI, and TSB. Sambrook et al., Molecular Cloning: A Laboratory Manual, 3rd edition, Cold Spring Harbor Press, Cold Spring Harbor, N.Y. (2001). An acidic medium may be selected to enrich a target microbe that is not a bacterium by determining the medium commonly used to grow the target microbe and acidifying that medium such that the target microbe gains a growth advantage.

The medium may be acidified through addition of numerous types of acids that include, but are not limited to, acetic acid, lactic acid, formic acid, hydrochloric acid, hydrofluoric acid, sulfuric acid, and phosphoric acid. Many types of organic and inorganic acids may be used to formulate the acidic media of the invention. Merck Index, 13^{th} edition, Merck & Co., Whitehouse Station, N.J. 2001. The pH of the acidic medium may be adjusted such that a selective advantage is conferred to the target microbe. A suitable pH may be determined for a given microbe by incubating the microbe in acidic media having differing pH, followed by incubation in growth media. The sample produced may be tested to determine conditions under which the population of a target microbe is increased relative to competitor microbes in the original sample. Examples of suitable pH ranges include those from about pH 1.5 to about 6, and single unit integers and fractions thereof. Other factors may be taken into account when selecting a suitable pH for the acidic medium. Such factors include, but are not limited to, the microbe being enriched, the time of incubation in the acidic medium, the temperature of incubation, the pressure under which incubation occurs, and the type of medium being used.

The amount of time that a sample is incubated in an acidic medium will depend on factors that include, but are not limited to, the pH of the acidic medium, the temperature of incubation, the target microbe being selectively enriched, competitor microbes present in the sample, and the type of medium that is used. Those of skill in the art can determine a time suitable for incubation of a sample containing a target microbe and competitor microbes in an acidic medium by determining the time that will kill or growth inhibit the competitor microbes while allowing the target microbe to replicate in a growth medium. Exemplary times include those from about one-half of an hour to about ten hours, and single unit increments and fractions thereof. For example, a sample can be enriched for E. coli O157:H7 by incubating the sample in an acidic medium at about pH 2 for about two to three hours and then allowing the E. coli O157:H7 to replicate in a growth medium. As indicated in Example I and II, such conditions decrease the number of competitor bacteria present in the test sample while allowing the target E. coli O157:H7 to replicate.

Numerous types of media suitable for growing microbes are well known in the art. Such media include, but are not limited to, GYT medium, LB medium, M9 minimal medium, NZCYM medium, NZYM medium, SOB medium, SOC medium, TB medium, 2×YT medium, BHI, and TSB. Sambrook et al., Molecular Cloning: A Laboratory Manual, 3rd edition, Cold Spring Harbor Press, Cold Spring Harbor, N.Y. (2001). Growth media may be selected to enrich a target microbe according to the invention that is not a bacterium by determining the media commonly used to replicate the target microbe. The growth media may be different than the acidic media used pursuant to the practice of the invention. Thus, following incubation of a sample in an acidic medium, the sample and acidic medium may be combined with the growth medium such that the target microbe replicates in the growth mediun. Alternatively, following incubation of a sample in the acidic medium, the pH of the acidic medium may be adjusted to produce growth medium. This modification may be achieved by adding a pH modifier to the acidic medium.

The temperature at which a sample containing target and competitor microbes is incubated will depend on the identity of the target microbe that is being enriched as well as the properties of the incubation medium. Generally, a target microbe may be incubated in an acidic medium at a temperature that is readily determined on one of skill in the art. Similarly, a target microbe may be incubated in growth medium at a temperature that mimics the temperature that the target microbe is normally found in nature. For example, a sample containing a target microbe that is an enteric bacterium found in association with a mammal may be independently incubated in an acidic medium at a suitable temperature that is dependant on the identity of the bacterium and the pH of the acidic medium, and in a growth medium at about 20 to 22° C. Examples of such microbes include, but are not limited to, E. coli, Shigella, and Listeria. In another example, a microbe may be incubated at a temperature that is about 37° C. or higher. Such a higher temperature may be in a range from about 38° C. to about 100° C. Examples of thermophilic microbes have been isolated from hot springs and deep-sea vents where the temperatures approach 100° C. Alternatively, a sample may be incubated at temperatures that range below 37° C, such as about 4° C. to about 36° C. Such lowered incubation temperatures may be suitable for enhancing microbes isolated from cold environments, such as arctic waters. In some embodiments it may be preferable to incubate the sample at temperatures that range from about 20° C. to about 22° C. Those of skill in the art will readily be able to determine the proper temperature for incubation of a sample in acidic and growth media.

Incubation of a sample in an acidic medium or a growth medium may be conducted independently under aerobic or anaerobic conditions. Thus, the methods and kits of the invention may be used to enhance or enrich a sample with a target microbe that grows under aerobic and anaerobic conditions.

A sample may be incubated in an acidic medium or a growth medium under ambient pressure, or under increased or decreased pressure. Such methods may be particularly useful for enriching microbes that are normally found in environments of high pressure, such as those that are found deep in the ocean.

Selective agents may optionally be included in the acidic medium, the growth medium or both. Such selective agents include, but are not limited to, antibiotics, phages, viruses, and nutrients.

The methods of the invention may be automated. For example, a first sample can be collected and placed into an acidic medium by a robotic arm. The first sample can be incubated in the acidic medium to produce a second sample. A robotic arm can then be used to transfer all or a portion of the second sample into a growth medium where the second sample can be incubated to enrich a microbe contained therein. All or a portion of the second sample may be transferred to a detector or another vessel where a microbe contained in the second sample can be detected.

In another example, a first sample may be incubated in an acidic medium to produce a second sample. The pH of the acidic medium can then be increased to produce a growth medium in which a microbe is enriched. An instrument, such as an automatic titrator, can be used to raise the pH of the acidic medium. This may be done by positioning the instrument such that the instrument can input a pH modifying agent into the acidic medium to produce a growth medium. The growth medium can then be incubated to enrich any target microbes contained therein. Instruments that can be used to alter the pH of media, such as automatic titrators, are commercially available. Analyticon Instruments Corp., Springfield, N.J; PAM Solutions LTD, Helsinki, Finland; Thermo Automation Systems, Beverly, Mass.

A variety of fermentors may also be used within the methods of the invention to enrich a target microbe. Such fermentors may be programmed to incubate a first sample at an acidic pH to produce a second sample, and then adjust the pH of the medium to produce a growth medium for enrichment of a microbe contained therein. Fermentors are commercially available. New Brunswick Scientific, Edison, N.J.

Automated methods are very useful for samples suspected of containing highly pathogenic microbes. Such methods may also be used for high-throughput screening of samples.

Kits

The invention provides kits that may be used to enrich a target microbe in a sample. The invention also provides kits that may be used to enrich and detect a target microbe in a sample.

A kit of the invention may include packaging material that contains an acidic medium or media in dry form. The acidic medium or media may be sterile or non-sterile. The packaging may or may not be capable of being sterilized. Examples of sterilization methods that may be used include, but are not limited to, baking, autoclaving, and irradiation. The packing material can be constructed to allow fluid to be combined with the dry acidic medium or media in the packaging material. In one example, the packaging material is a container containing the dried acidic medium into which fluid may be added to form a liquid medium. The packaging material containing the liquid medium may be sterilized, inoculated with a sample, and incubated. In another example, the packing material containing the acidic medium may be sterilized prior to addition of sterile fluid to provide a container that may be inoculated with a sample and incubated.

The invention provides a kit that includes packaging material and sterile acidic media or medium in liquid form. The sterile acidic media or medium may be transferred to another container, inoculated with a sample, and incubated. Alternatively, the sterile acidic medium in the packaging material may be inoculated and incubated.

A kit of the invention may include packaging material that contains individual packages of acidic media and growth media in dried form. The individual packages containing acidic and growth media may or may not be sterile. The dried acidic and growth media may be combined with fluid in a separate container, or combined with fluid in the packaging material to form liquid media. The packaging material may or may not be able to be sterilized. Packaging material containing liquid media may optionally be inoculated with a sample and incubated.

A kit of the invention may include an acidic medium or a growth medium, and one or more pH modifiers. An example of a pH modifier included in the kit may be a basic pH modifier, such as sodium hydroxide, or an acidic pH modifier, such as hydrochloric acid. The acidic medium may be made into a growth medium by addition of a basic pH modifier.

The invention provides a kit having packaging material that includes a liquid or a dry medium or media, an acidic pH modifier, and a basic pH modifier. An example of such a kit includes packaging material containing a liquid medium that has been sterilized, a sterile acidic pH modifier, and a sterile basic pH modifier. The acidic pH modifier may be combined with the liquid medium to form acidic medium which is inoculated with a sample and incubated. The basic pH modifier is then added to the incubated acidic medium to form growth medium which is then incubated to enrich a target microbe present in the sample. Such a kit may contain one or more acidic pH modifiers and one or more basic pH modifiers to allow one of skill in the art to select the pH of the acidic medium and the growth medium. In another example, the kit includes packaging material that contains a sterile acidic medium and a basic pH modifier. The acidic medium contained within the packaging material may be inoculated with a sample and incubated. The basic pH modifier may be added to the incubated acidic medium to form growth medium which may be incubated to enrich a target microbe present in the sample.

In another example, the kit includes packaging material that contains a sterile liquid acidic medium having a pH of about 2, and a sterile liquid growth medium that has a pH of about 7 after addition of the acidic medium contained within the kit.

A kit of the invention may include packaging material that contains an acidic medium, a growth medium, and a means for detecting a target microbe. The acidic medium and the growth medium may optionally be dry or fluid. The acidic medium and the growth medium may be optionally sterile if in dry form. The acidic medium and the growth medium may be packaged in sterile fluid form. Numerous types of detection means may be included in the kit. Examples of such detection methods are described herein and are available to those of skill in the art. These detection methods include, but are not limited to, indicator media, immunological based methods, and nucleic acid detection and amplification assays.

Detection Methods

The methods of the invention may be used in combination with numerous detection methods. These detection methods can be tailored to detect the presence of a broad class of microbes in a sample, or used to detect specific microbes in a sample. An advantage of the methods of the present invention is that a microbe may be selected and enriched in the absence of selective agents other than decreased pH. Therefore, the present invention may be used to avoid interference by a selective agent during the detection phase of an assay used to determine the presence of a microbe in a sample.

Enzyme linked immunosorbant assays (ELISA) are commonly used to detect microbes in a sample. Such assays generally involve immobilizing a microbe to a solid support and then adding an antibody that is conjugated with an enzyme to the immobilized microbe. The conjugated antibody binds to an antigen on the microbe to form a complex. A substrate is then contacted with the complex such that the enzyme that is conjugated to the antibody converts the substrate into a detectable product. Many such assays are available and known in the art. For example, an ELISA based assay has been used to detect E. coli O157:H7 in raw meat and in stool samples. Johnson et al., Appl. Envir. Microbiol., 61:386 (1995); Bennett et al., Lett. App. Microbiol., 20:375 (1995); Dylla et al., J. Clin. Microbiol. 33:222 (1995). ELISA based assays have also been used to detect Shigella like toxin-1 and Shigella like toxin-2 in stool samples. Yamada et al., Microbiol. Immunol., 37:111 (1993). Those of skill in the art will realize that numerous ELISA based assays may be used in combination with the methods and kits of the invention to detect a plethora of microbes in a large variety of samples.

It is known that the results of enzyme linked immunosorbant assays are greatly influenced by acriflavine, bile salts, and heat. Johnson et al., Appl. Envir. Microbiol., 61:386 (1995). Thus, because the standard methods currently used by the FDA to enrich microbes in a sample include antibiotics and bile salts, these methods may not be compatible with ELISA based assays. Accordingly, the methods and kits of the present invention offer the advantage that they can be used in combination with detection methods that are affected by the presence of selective agents, such as bile salts and antibiotics.

Many other types of assays utilizing antibodies have been developed to detect the presence of a microbe in a sample. Examples of such methods include, but are not limited to, direct immunofluorescent antibody staining to detect the microbe. These types of detection methods have been used to detect E. Coli O157:H7 in stool samples. Park et al., Am. J. Clin. Pathol., 101:91 (1994). Phenotypic variants of serotype O157:H7 retain the O157 antigen; hence antibodies to O157 antigen can be used to identify both serotype O157:H7 and its variants. In the clinical laboratory, anti-O157 sera are used effectively in agglutination or latex agglutination tests to rapidly screen or serologically confirm isolates. Some anti-O157 antibodies have also been coupled to magnetic beads and used to selectively isolate this pathogen from foods or have been incorporated into enzyme immunoassays to directly detect serotype O157:1H7 in foods and clinical specimens. Wright et al., Epidemiol. Infect., 113:31 (1994).

Nucleic acid amplification can also be used to detect the presence of a target microbe in a sample. Any such amplification procedure can be used, for example, polymerase chain reaction (PCR) assays, strand displacement amplification and other amplification procedures. Strand displacement amplification can be used as described in Walker et al (1992) Nucl. Acids Res. 20, 1691-1696. The term “polymerase chain reaction” (“PCR”) refers to the method of K. B. Mullis U.S. Pat. Nos. 4,683,195; 4,683,202; and 4,965,188, hereby incorporated by reference, which describe a method for increasing the concentration of target nucleic acid in a mixture of genomic DNA or other DNA or RNA without cloning or purification.

The steps involve in PCR nucleic acid amplification method are described in more detail below. For ease of discussion, the nucleic acid to be amplified is described as being double-stranded. However, the process is equally useful for amplifying a single-stranded nucleic acid, such as an MRNA, although the ultimate product is generally double-stranded DNA. In the amplification of a single-stranded nucleic acid, the first step involves the synthesis of a complementary strand so that two complementary target strands are available for amplification.

When PCR is performed on a nucleic acid present in a target microbe, two primers are employed, each primer hybridizing to a different nucleic acid strand at opposite ends of the nucleic acid site to be amplified. The PCR process for amplifying a target nucleic acid consists of introducing a large excess of the two primers to a mixture that may contain a template nucleic acid from a target microbe, followed by a precise sequence of thermal cycling in the presence of a nucleic acid polymerase. For PCR amplification, each of the two primers is complementary to a distinct region in one of the two strands of the double stranded target sequence. Primers are selected so that they hybridize just outside the region of interest to be amplified and so that, upon primer extension, one primer will be extended towards the hybridization site of a second primer hybridized on the opposite target strand.

To effect amplification, the template DNA is denatured to open up double-stranded target sites and the temperature is lowered so that the primers anneal to their complementary sequences within the template. Following annealing, the primers are extended with a polymnerase. Such primer extension forms a new pair of complementary strands that likely have different ends than the original target. Such complementary strands can hybridize together to form an “amplicon” that can also be a target for amplification. The steps of denaturation, primer annealing and primer extension can be repeated many times. Each round of denaturation, annealing and extension constitutes one “cycle.” There can be numerous cycles, and the amount of amplified DNA produced increases with the number of cycles. Hence, to obtain a high concentration of an amplified target nucleic acid, many cycles are performed.

For example, polymerase chain reaction (PCR) mismatch amplification assays can be used to detect a target microbe in a sample. Such methods have been used to specifically amplify and detect the uidA gene that is unique to E. coli O157:H7. Feng, Mol. Cell. Probes, 7:151 (1993).

Several methods have been developed for isolation of E. coli O157:H7 from food and clinical samples based on the knowledge that it is unable to ferment sorbitol during standard incubation periods or to produce beta-glucuronidase. Farmer et al., J. Clin. Microbiol., 22:620 (1985). Accordingly, E. coli O157:H7 has been detected through use of a methylumbelliferyl glucuronide assay that measures glucuronidase activity. Feng et al., Appl. Environ. Microbiol., 43:1320 (1982). Based on the inability of E. coli O157:H7 to ferment sorbitol, a number of selective and differential plating media have been developed that indicate the presence of E. coli O157:H7. For example, sorbitol MacConkey agar (SMAC; Difco Laboratories) is routinely used as a selective, differential plating medium. E. coli O157:H7 produce near colorless colonies when grown on sorbitol MacConkey agar because they are unable to ferment sorbital, whereas other bacteria that are able to ferment sorbitol produce red colonies. However, the color changes are not strongly differentiable. Sometimes the red color produced by other bacteria can cover the nearly colorless E. coli O157:H7 colonies, so they can be missed. Recently, a new medium called 202 medium was developed. Fung et al., Proc. Food Safety Cons., annual meeting proceedings, Kansas City, Mo. (1997). E. coli produced a yellow color in this greenish blue medium, whereas E. coli O157:H7 produced a green color.

Another medium was developed that for differentiation between E. coli and E. coli O157:H7. This medium was prepared by comparing several combinations of dyes that can be used to differentiate E. coli from E. coli O157:H7. Some studies indicate that indigo carmine and phenol red are a good combination. These dyes were added to basal agar (SMAC medium excluding neutral red and crystal violet) to produce EOH medium. On the dark blue EOH medium, E. coli produced a yellow color with a clear zone, whereas E. coli O157:H7 produced a red color without a clear zone. Isolation of serotype O157:H7 from samples, such as foods, in selective media, such as hemorrhagic colitis agar and cefizime-MacConkey agar, is used in the clinical laboratory as the primary screening medium to analyze patient specimens for the presence of serotype O157:H7. Szabo et al., J. Food Prot., 49:768 (1986); March and Ratam, J. Clin. Microbiol., 23:869 (1986).

Although very useful, isolating and identifying the pathogen exclusively on the absence of sorbitol fermentation has limitations. Other enteric bacteria, such as E. hermanii and Hafnia spp., share similar phenotypes and resemble serotype O157:H7 on sorbitol-containing medium. Likewise, non-H7 serotype strains of O157 that are not pathogenic but do not ferment sorbitol have occasionally been isolated from foods. Willshaw et al., J. Appl. Bacteriol., 75:420 (1993). Because of the presence of phenotypically similar species, sorbitol negative isolates should be serologically confirmed with O157 and H7 antisera.

It is understood that the methods and kits of the invention may be used in combination with numerous methods to detect the presence of microbes in many different types of samples. Examples of different types of samples include, but are not limited to: environmental samples such as water; foods such as meats, vegetables, fruits, and the like; and bodily samples obtained from the bodies of animals and humans, such as blood, urine, stool, skin, mucus, saliva, tears, and the like.

The invention will be further described by reference to the following detailed examples, which are given for illustration of the invention, and are not intended to be limiting thereof.

### EXAMPLE I

### Enrichment of Enterohemorragic E. coli using Standard FDA Methods Compared to Enrichment using Methods of the Invention with Fresh Bacterial Cultures.

A sample containing enterohemorragic E. coli was enriched through use of the standard FDA method according to the following protocol. Cultures containing individual strains of enterohemorrhagic E. coli were diluted to levels of <1000 cells/ml and added to mTSB plus cefixime, cefsulodin, and vancomycin. Cultures were incubated for 24 hours at 35° C. The concentration of the cells was then determined using standard decimal dilution methods followed by spread plating on TSAYE agar. Note that standard methods call for incubation at 37° C. with shaking, but stationary incubation at 35° C. was found to be better for accurate monitoring of large numbers of cultures (U.S.F.D.A. Bacteriological Analytical Manual-fda.gov).

Other samples containing enterohemorragic E. coli were enriched according to one of the methods of the invention. A first sample containing an enterohemorragic E. coli O157:H7 culture, or one of the other nine competitor bacterial cultures, were individually added to containers having an acidic medium (pH 2.0) and incubated at 22° C. for two hours to produce second samples of each. The components of the acidic medium included Tryptic Soy Broth (TSB, Difco) that was adjusted to pH 2.0. Following incubation in the acidic medium, each second sample was transferred to a growth medium containing TSB that was adjusted to near neutral pH and incubated at 35° C. for 24 hours to produce a test sample. Following incubation in the growth medium, the populations of enterohemorrhagic E. coli and each of the competing bacteria in the test samples were determined. The concentration of the enterohemorrhagic E. coli, and each of the nine competitor strains, was determined by using standard decimal dilution methods and spread plating on TSAYE. TSAYE plates were incubated for 24 hours at 35° C. Colonies on the plates were then counted and cell concentrations were calculated by multiplying the number of colonies by the dilution factor.

The number of bacteria of each type in the test sample prepared by the standard FDA method and the method of the invention are provided in Tables 1-3. The results indicate that the method of the invention gives better suppression of competitor bacteria than the standard FDA method.

TABLE 1

| | Enrichment | Enrichment | |
|---|---|---|---|
| | according to | according to a | |
| | standard | method of the | |
| | FDA method | invention as | |
| | (cells | described in | Method that |
| Competitor | per milliliter | Example I (cells per | provided the best |
| Bacteria | of culture) | milliliter of culture) | result |
| M morganii | 7.2 × 10⁷ | 7.8 × 10⁸ | Standard method |
| C. freundii | 3.3 × 10⁸ | <10 | Inventive method |
| E. aerogenes | 3.11 × 10⁸ | <10 | Inventive method |
| K. pneumoniae | 2.1 × 10³ | <10 | Inventive method |
| P. mirabilis | <10 | <10 | Comparable |
| E. sakazaki | 7.0 × 10⁸ | <10 | Inventive method |
| H. alvei | 5.6 × 10⁷ | 1.7 × 10⁹ | Standard method |
| E. Americana | <10 | <10 | Comparable |
| A. calcoaceticus | 9.13 × 10⁸ | <10 | Inventive method |

The results provided in Tables 2 and 3 show the number of enterohemorragic E. coli that were present in one milliliter of media following enrichment using one of the methods of the invention (Table 2) and the standard enrichment method used by FDA (Table 3). The results indicate that the methods of the invention enrich enterohemorragic E. coli to higher levels than standard methods currently used by FDA by factors ranging from about 1.6-fold to about 4.3-fold.

TABLE 2

| Strain of enterohemorrhagic | Number of cells |
|---|---|
| E. coli assayed in medium | contained in one |
| described in Example I. | milliliter of medium |
| 6424 | 10.3 × 10⁸ |
| 6443 | 9 × 10⁸ |
| 6457 | 6.75 × 10⁸ |
| 3579 | 5.63 × 10⁸ |
| 5506 | 4.88 × 10⁸ |
| 3039 | 8.5 × 10⁸ |
| 6321 | 14.13 × 10⁸ |
| 6347 | 10.25 × 10⁸ |
| 6396 | 12.13 × 10⁸ |
| 178190 | 8 × 10⁸ |

TABLE 3

| Strain of enterohemorrhagic E. coli | Number of cells contained |
|---|---|
| assayed in standard medium | in one milliliter of medium |
| 6424 | 2.7 × 10⁸ |
| 6443 | 2.5 × 10⁸ |
| 6457 | 1.79 × 10⁸ |
| 3579 | 3.64 × 10⁸ |
| 5506 | 1.71 × 10⁸ |
| 3039 | 3.6 × 10⁸ |
| 6321 | 3.25 × 10⁸ |
| 6347 | 3.63 × 10⁸ |
| 6396 | 3.13 × 10⁸ |
| 178190 | 4.25 × 10⁸ |

### EXAMPLE II

### Enterohemorragic E. coli Enrichment with Standard FDA Methods Compared to Enrichment According to the Method of the Invention using Age-Stressed Bacterial Cultures.

A sample containing enterohemorragic E. coli was enriched through use of the standard FDA method according to the protocol described in Example I.

Anther sample containing enterohemorragic E. coli was enriched according to one of the methods of the invention. A first sample containing individual age-stressed enterohemorragic E. coli O157:H7 cultures or nine age-stressed competitor bacterial cultures were individually added to an acidic medium (pH 2.0) and incubated at 22° C. for two hours to produce second samples. The components of the acidic medium included TSB adjusted to pH 2.0. Following incubation in the acidic medium, the second sample was transferred to non-inhibitory growth medium containing TSB at near neutral pH and incubated at 35° C. for 24 hours to produce a test sample. Following incubation in the growth medium, the populations of each of the enterohemorrhagic E. coli and each of the competing bacteria in the test sample was determined as described in Example I.

The number of bacteria of each type in the test sample prepared by the standard FDA method and the method of the invention are indicated below. The results indicate that the method of the invention gives better suppression of competitor bacteria than the standard FDA method. Another aspect of the method of the invention is that it provided better results with the age-stressed bacterial culture than with the fresh bacterial culture. This is thought to more closely reflect conditions occurring with a clinical, food, or water sample obtained for testing and analysis.

TABLE 4

| | Enrichment | Enrichment by | |
|---|---|---|---|
| | by | the inventive | |
| | standard FDA | method described | Method that |
| Competitor | method | in Example | provided the best |
| Bacteria | (cells/ml) | I (cells/ml) | result |
| M morganii | 9.8 × 10⁷ | <10 | Inventive method |
| C. freundii | 2.1 × 10⁸ | <10 | Inventive method |
| E. aerogenes | 2.3 × 10⁸ | <10 | Inventive method |
| K. pneumoniae | 1 × 10³ | <10 | Inventive method |
| P. mirabilis | <10 | <10 | Comparable |
| E. sakazaki | 9.6 × 10⁸ | <10 | Inventive method |
| H. alvei | 9.8 × 10⁷ | 1.3 × 10⁹ | Standard method |
| E. americana | <10 | <10 | Comparable |
| A. calcoaceticus | 4.3 × 10⁸ | <10 | Inventive method |

The results provided in Tables 5 and 6 show the number of enterohemorragic E. coli that were present in one milliliter of medium following enrichment using one of the methods of the invention (Table 5) and the standard enrichment method used by FDA (Table 6). The results indicate that the methods of the invention enrich enterohemorragic E. coli to higher levels than standard methods currently used by FDA by factors ranging from about 2.6-fold to about 6-fold.

TABLE 5

| Strain of enterohemorrhagic E. coli | Number of cells |
|---|---|
| assayed in the medium according to | contained in one |
| Example II after being aged for 44 days | milliliter of medium |
| 6424 | 10 × 10⁸ |
| 6443 | 9.25 × 10⁸ |
| 6457 | 12.38 × 10⁸ |
| 3579 | 8.75 × 10⁸ |
| 5506 | 7.5 × 10⁸ |
| 3039 | 7.5 × 10⁸ |
| 6321 | 10.25 × 10⁸ |
| 6347 | 7.38 × 10⁸ |
| 6396 | 9.25 × 10⁸ |
| 178190 | 5.25 × 10⁸ |

TABLE 6

| Strain of enterohemorrhagic E. coli | Number of cells |
|---|---|
| assayed in standard medium after | contained in one |
| being aged for 44 days | milliliter of medium |
| 6424 | 2.25 × 10⁸ |
| 6443 | 0.83 × 10⁸ |
| 6457 | 2.25 × 10⁸ |
| 3579 | 1.01 × 10⁸ |
| 5506 | 1.14 × 10⁸ |
| 3039 | 2.09 × 10⁸ |
| 6321 | 1.88 × 10⁸ |
| 6347 | 2.95 × 10⁸ |
| 6396 | 1.99 × 10⁸ |
| 178190 | 2.03 × 10⁸ |

### EXAMPLE III

### Acid-Selection Treatment.

Cell of 13 species were placed into one of 10 acid treatments for 2 hours at room temperature (about 22° C.), and then transferred to noninhibitory medium (TSB) for incubation at 35° C. for 24 hours. If growth was seen at 24 hours, the result was listed as a positive (pos). If no growth was seen at 24 hours, the result was listed as negative (neg). Seven of the treatments were TSB adjusted to a pH of 1 to 7. The eighth treatment was TSB (pH 2), plus 1% sodium glutamate. The ninth and tenth treatments were Brain Heart Infusion (pH 2) and Luria Broth (pH 2) respectively. The species used were Salmonella gaminara, Shigella flexneri Staphylococcus aureus, Pseudomonas aeruginosa, Klebsiella pneumoniae, and Listeria monocytogenes, nonpathogenic E. coli (EC6268), enterohemorrhagic E. coli (EC6264) Citrobacter freundii, Hafnia alvei, Morganella morganii, Enterobacter aerogenes, and Serratia marcescens.

TABLE 7

| | | | | Acid-Selection Treatment | | | | TSB | | |
|---|---|---|---|---|---|---|---|---|---|---|
| | TSB | TSB | TSB | TSB | TSB | TSB | TSB | Glutamate | BHI | LB |
| Species | pH 1.0 | pH 2.0 | pH 3.0 | pH 4.0 | pH 5.0 | pH 6.0 | pH 7.0 | pH 2.0 | pH 2.0 | pH 2.0 |
| Salmonel. | neg | neg | pos | pos | pos | pos | pos | neg | neg | neg |
| Shigella | neg | variable | pos | pos | pos | pos | pos | pos | pos | pos |
| Staphylo. | neg | neg | pos | pos | pos | pos | pos | neg | neg | neg |
| Pseudom. | neg | neg | neg | pos | pos | pos | pos | neg | neg | neg |
| Klebsiell. | neg | neg | pos | pos | pos | pos | pos | neg | neg | neg |
| Listeria | neg | neg | pos | pos | pos | pos | pos | neg | neg | neg |
| EC6268 | neg | pos | pos | pos | pos | pos | pos | pos | pos | pos |
| EC6424 | neg | pos | pos | pos | pos | pos | pos | pos | pos | pos |
| Citrobact. | neg | neg | neg | pos | pos | pos | pos | neg | neg | neg |
| Hafnia | neg | pos | pos | pos | pos | pos | pos | neg | pos | pos |
| Morganel. | neg | neg | pos | pos | pos | pos | pos | neg | neg | neg |
| Enteroba. | neg | neg | pos | pos | pos | pos | pos | neg | neg | neg |
| Serratia | neg | neg | pos | pos | pos | pos | pos | neg | neg | neg |

### EXAMPLE IV

### Growth of Cells Exposed to TSB (pH 2.0) at Room Temperature

Cells of nine species were placed into TSB (pH 2.0) for various lengths of time at room temperature (about 22° C.). The cells were then transferred to noninhibitory growth medium (TSB) for incubation at 35° C. for 24 hours. If growth was seen at 24 hours, the result is listed as positive (pos). If no growth was seen at 24 hours, the result is listed as negative (neg). The species used were Citrobacter freundii, Hafnia alvei, Shigella flexneri, Enterobacter aerogenes, Klebsiella pneumoniae, EC6347, EC6396, EC6424 and EC6268. The EC6347, EC6396 and EC6424 strains are all pathogenic strains of E. coli, but EC6268 is a nonpathogenic strain of E. coli.

TABLE 8
Length of Time Cells were Exposed to pH 2.0 Using TSB (pH2.0)
at room temperature (about 22° C.)

| Species | 1 hour | 2 hours | 3 hours | 4 hours | 5 hours |
|---|---|---|---|---|---|
| Citrobact. | Neg | Neg | neg | neg | neg |
| Hafnia | Pos | Pos | neg | neg | neg |
| Shigella | Pos | Neg | neg | neg | neg |
| Enteroba. | Neg | Neg | neg | neg | neg |
| Klebsiell. | Neg | Neg | neg | neg | neg |
| EC6347 | Pos | Pos | pos | pos | pos |
| EC6396 | Pos | Pos | pos | pos | pos |
| EC6424 | Pos | Pos | pos | pos | pos |
| EC6268 | Pos | Pos | pos | pos | pos |

### EXAMPLE V

### Growth of Cells Exposed to TSB (pH 2.0) at Various Temperatures

Cells of six species were placed into TSB (pH 2.0) for 2 hours at various temperatures. The cells were then transferred to noninhibitory growth medium (TSB) for incubation at 35° C. for 24 hours. If growth was seen at 24 hours, the result was listed as positive (pos). If no growth was seen at 24 hours, the result was listed as negative (neg). The species used were Citrobacter freundii, Hafnia alvei, EC6396, EC6424 and EC6268. The EC6396 and EC6424 strains are pathogenic E. coli. EC6268 is non-pathogenic; and Shigella flexneri. Note, the initial cell counts for Shigella flexneri were lower than expected. Thus, the negative growth of Shigella at 5° C. and 22° C. (#1) may have been due to absence of cells.

TABLE 9
Variations in Temperature as Cells are
Exposed to TSB (pH 2.0) for 2 hours

| Species | 5° C. | 22° C.(RT) #1 | 22° C. #2 | 35° C. | 45.5° C. |
|---|---|---|---|---|---|
| Citrobact. | Neg | Neg | neg | neg | neg |
| Hafnia | Neg | Pos | neg | neg | neg |
| EC6396 | Pos | Pos | pos | pos | neg |
| EC6424 | Pos | Pos | pos | neg | neg |
| EC6268 | Pos | Pos | pos | neg | neg |
| Shigella | Neg | Neg | pos | neg | neg |

### EXAMPLE VI

### Improved Ability to Conduct PCR Analysis with the Acid Enrichment Method

This Example illustrates another way in which the new acid enrichment method is superior to the current FDA accepted methods for potential rapid detection of E. coli O157:H7 and other enterohemorrhagic E. coli. The current FDA method requires a 24 hr enrichment in enterohemorrhagic E. coli enrichment broth (EEB) as prescribed in the U.S. FDA-Bacteriological Analytical Manual (FDA-BAM), then streaking onto TCSMAC agar and incubation for another 24 hr. MacConkey Sorbitol Agar with Cefixime and Tellurite (TCSMAC) is a selective medium used for the isolation and differentiation of Escherichia coli O157:H7.

However, emerging trends in microbiology require shorter analytical procedures, for example, those based on PCR. Hence, a PCR procedure that can be used on the initial enrichment broth culture was developed that did not require streaking onto selective agar and incubation for another 24 hr. Using this PCR-based procedure with the acid enrichment procedures of the invention can reduce total analytical time to about 27 hours (24 hr for incubation, plus 2 or 3 for PCR) vs. 48 hr for the FDA procedure.

A sample of wastewater was obtained to provide numerous types of competing bacteria and this wastewater sample was spiked with a known culture of enterohemorrhagic E. coli. The sample was acid-shocked in pH 2.00 TSB for 2 hr, then transferred to TSB at regular pH (7.4) for 24 hr of growth. For comparison, a similar sample of the wastewater/enterohemorrhagic E. coli was cultured in EEB according to the usual FDA procedure and incubated 24 hr. At the end of the 24 hr period, one ml aliquots were withdrawn from each of the experimental and control cultures. These test aliquots were centrifuged twice to remove contaminants and boiled to provide PCR templates.

PCR reactions were set up using primers for three genes characteristic of the presence of enterohemorrhagic E. coli—glutamate decarboxylase (gad), shiga-like toxin 1 (st×1) and shiga-like toxin 2 (st×2). PCR procedures were performed as described in Grant et al. (2001) Applied and Environmental Microbiology 67:3110-14. The resulting amplicons were run on a 1.5% agarose gel and visualized with ethidium bromide.

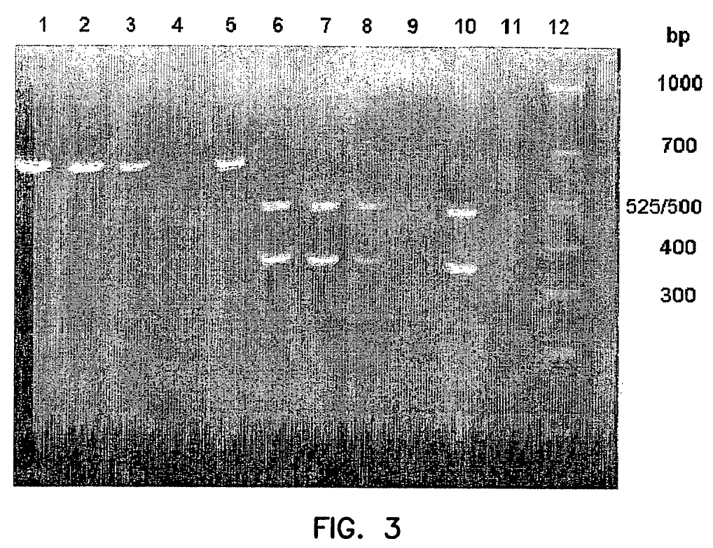

The results are shown in FIG. 3. Lanes 1 and 2 show that bands typical of the gad gene that is a marker for enterohemorrhagic E. coli were generated using two versions of the inventive acid enrichment method. Lane 4 shows no band for the gad gene—the template for this amplification reaction was generated from FDA accepted enrichment procedures. Lanes 3 and 5 are positive controls showing that the gad fragment is generated under the PCR conditions employed when enterohemorrhagic E. coli DNA is used as template.

Lanes 6 and 7 show that bands typical of the st×1/2 genes were generated from samples treated by the inventive acid enrichment method. Lane 9 shows that no PCR band is generated when microbial samples are enriched as specified by FDA procedures (BAM EEB). Lanes 8 and 10 were positive controls showing that the gad fragment is generated under the PCR conditions employed when enterohemorrhagic E. coli DNA is used as template.

In summary, templates prepared from bacteria cultured using the acid enrichment methods of the invention provided nucleic acid templates that were readily amplified by PCR to generate amplicons indicative of three enterohemorrhagic E. coli template target genes. In contrast, something in the FDA accepted (EEB) medium interfered with PCR detection of enterohemorrhagic E. coli. These data indicate that there are one or more component(s) in the EEB solution that interfere with PCR.

While streaking to selective TCSMAC agar is required under FDA procedures at this point, workers may be tempted to speed up the FDA accepted assay by employing PCR procedures. However, if a bacteriologist was to use the FDA accepted EEB enrichment procedure and then employ PCR, the bacteriologist would get a false negative result. Such a result could frustrate and delay appropriate handling of contaminated water or food sources, thereby endangering workers and consumers with further exposure to harmful pathogens.

Hence, the methods of the invention that involve strong acid shock, followed by growth in completely non-inhibitory media such as regular pH TSB generates nothing that inhibits or interferes with PCR analysis. Thus, microbiologists can effectively screen for enterohemorrhagic E. coli in only 25-26 hr using PCR, instead of waiting an additional day or more for results on selective agar as is now required under FDA accepted procedures. Moreover, such a PCR approach would be useful for detection of many types of pathogenic E. coli, such as enterotoxigenic E. coli (ETEC), enteropathogenic E. coli (EPEC) and others, depending on the target genes selected.

### EXAMPLE VII

### Improved Enrichment of E. coli O157:H7 by the Acidification Method of the Invention

This Example illustrates that the enrichment procedures described herein are superior to several enrichment procedures currently employed by several governmental regulatory agencies.

A sample of wastewater (i.e. untreated sewage) was collected to provide a wide variety of competing microorganisms. This sample was spiked with E. coli O157:H7 (strain SEA 6424). Aliquots were then removed from this common mixture and enriched by the acidification method of the invention and by four other methods commonly used by various governmental regulatory agencies—the current FDA method (BAM), the current USDA method, the current Canadian method and Buffered Peptone Water, a commonly used non selective medium.

For the acidification method, the aliquot sample was placed in pH 2.00 TSB for 2 hr at room temperature then transferred to TSBYE for 24 hr.

The USDA method employed is described in the USDA Microbiology Laboratory Guidebook (http://www.fsis.usda.gov/ophs/microlab/mlgbook.htm). Briefly, an aliquot of the E. coli O157:H7-spiked wastewater was placed in modified EC broth with novobiocin and incubated for 24 h at 35° C.

The Canadian method employed is described in Government of Canada, Health Products and Food Branch, Ottawa, Isolation of E. coli O157:H7 in Foods (http://www.hc-sc.gc.ca/food-aliment/mh-dm/mhe-dme/compendium/e_index.html). Briefly, an aliquot of the E. coli O157:H7-spiked wastewater was placed into modified tryptic soy broth (mTSB) with novobiocini and incubated for 24 h at 42° C. Note that the Canadian method also permits use of EHEC enrichment broth (EEB), whose performance was examined as described for the FDA method below.

The FDA method employed is described in the FDA Bacteriological Analytical Manual Online, 2002, chap. 4A (http://www.cfsan.fda.gov/˜ebamn/bam-toc.html). Briefly, an aliquot of the E. coli O157:H7-spiked wastewater was placed into EHEC Enrichment Broth (mTSB with cefixime, cefsulodin and vancomycin, but without novobiocin) and then incubated at 35° C. for 24 hr. Note that this is a slight modification of the BAM method that specifies 37° C. with shaking.

At the end of each of the enrichment periods, the cultures were plated onto three standard selective agars—TCSMAC, Rainbow agar and Rainbow agar plus tellurite and novobiocin. These three selective agars are the primary selective agars used world wide to see if enrichment has yielded any E. coli O157:H7 from a sample.

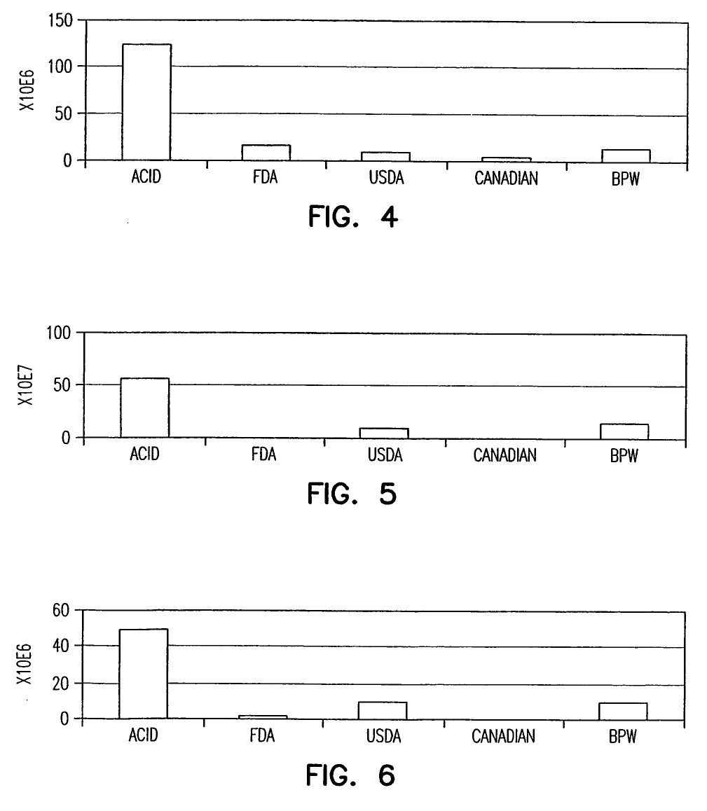

The results are provided in FIGS. 4-6. FIG. 4 provides the numbers of sorbitol negative colonies on TCSMAC plates. As shown, the acid enrichment yielded almost ten-fold more E. coli O157:H7 colonies relative to the other enrichment procedures (FIG. 4). FIG. 5 provides the number of black colonies on Rainbow agar. As shown, the acid enrichment again yielded five- to ten-fold more E. coli O157:H7 colonies relative to the other enrichment procedures (FIG. 5). FIG. 6 provides the number of black colonies on Rainbow agar containing tellurite and novobiocin. As shown, the acid enrichment again yielded five- to ten-fold more E. coli O157:H7 colonies relative to the other enrichment procedures (FIG. 6). Hence, the acid enrichment procedures of the invention provide larger recoveries of target colonies than any of the other enrichment methods, as tested by three different selective growth agars. These data indicate that acid enrichment method of the invention outperforms any of the methods currently used by governmental regulatory agencies.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A method comprising: incubating a first sample suspected of containing one or more competitor microbes and one or more target microbes in an acidic medium to produce a second sample; wherein incubating the first sample in the acidic medium can generate a second sample that has a higher percentage of non-pathogenic or pathogenic Escherichia coli than the first sample.

2. The method of claim 1, wherein the pathogenic Escherichia coli is enterohemorrhagic Escherichia coli, enteropathogenic Escherichia coli, or enterotoxigenic Escherichia coli.

3. The method of claim 1, wherein one or more competitor microbes is a competitor bacterium.

4. The method of claim 3, wherein one or more competitor bacteria are killed or growth inhibited during incubation in the acidic medium.

5. The method of claim 1, wherein one or more target microbes is selected from the group consisting of Salmonella, Shigella, Staphylococcus, Klebsiella, Escherichia, Listeria, Morganella, Enterobacter, Serratia, Yersinia, Bacillus and Hafnia.

6. The method of claim 1, wherein one or more target microbes is a pathogenic bacterium.

7. The method of claim 6, wherein the pathogenic bacterium is enterohemorrhagic Escherichia coli, enteropathogenic Escherichia coli, or enterotoxigenic Escherichia coli.

8. The method of claim 6, wherein the pathogenic bacterium is Escherichia coli O157:H7.

9. The method of claim 1, wherein the pH of the acidic medium is between 1 and 6.

10. The method of claim 1, wherein the pH of the acidic medium is between 2 and 4.

11. The method of claim 1, wherein the pH of the acidic medium is between 2 and 3.

12. The method of claim 1, wherein the first sample is an environmental sample.

13. The method of claim 1, wherein the first sample is a water sample.

14. The method of claim 1, wherein the first sample is a food sample.

15. The method of claim 1, wherein the first sample is a bodily sample.

16. The method of claim 1, wherein the acidic medium is selected from the group consisting of GYT medium, LB medium, M9 minimal medium, NZCYM medium, NZYM medium, SOB medium, SOC medium, TB medium, 2× YT medium, BHI, and TSB.

17. The method of claim 1, wherein the acidic medium comprises a selective agent.

18. The method of claim 17, wherein the selective agent is an antibiotic.

19. The method of claim 17, wherein the selective agent is a bacteriophage.

20. The method of claim 17, wherein the selective agent is a nutritional supplement.

21. The method of claim 17, wherein the selective agent is an inorganic selective agent.

22. The method of claim 17, wherein the selective agent is an organic selective agent.

23. The method of claim 17, wherein the selective agent is tellurite, selenite or sorbitol.

24. The method of claim 1, wherein the first sample is incubated in the acidic medium for 0.1 to 10 hours.

25. The method of claim 1, wherein the first sample is incubated in the acidic medium for 1 to 4 hours.

26. The method of claim 1, wherein the first sample is incubated in the acidic medium for 1 to 2.5 hours.

27. The method of claim 1, wherein the first sample is incubated in the acidic medium at a temperature that is about 5° C. to about 20° C.

28. The method of claim 1, wherein the first sample is incubated in the acidic medium at a temperature that is about 20° C. to about 45° C.

29. The method of claim 1, wherein the first sample is incubated in the acidic medium at a temperature that is about 20° C. to 22° C.

30. The method of claim 1, wherein the first sample is incubated in the acidic medium at a temperature that is between 37° C. and 70° C.

31. The method of claim 1, wherein the acidic medium comprises glutamate.

32. The method of claim 1, further comprising detecting one or more target microbes in the second sample.

33. The method of claim 32, wherein one or more target microbes is detected using selective growth media.

34. The method of claim 33, wherein the selective growth media comprises an antibiotic.

35. The method of claim 33, wherein the selective growth medium comprises a bacteriophage.

36. The method of claim 33, wherein the selective growth medium comprises a nutritional supplement.

37. The method of claim 33, wherein the selective agent is an inorganic selective agent.

38. The method of claim 33, wherein the selective agent is an organic selective agent.

39. The method of claim 33, wherein the selective growth medium comprises tellurite, selenite, or sorbitol.

40. The method of claim 33, wherein the selective growth medium comprises a medium selected from the group consisting of GYT medium, LB medium, M9 minimal medium, NZCYM medium, NZYM medium, SOB medium, SOC medium, TB medium, 2× YT medium, BHI, and TSB.

41. The method of claim 32, wherein one or more target microbes is detected using antibodies directed against the target microbe, enzyme-linked immunosorbent assay, or radioimmunoassay.

42. The method of claim 32, wherein one or more target microbes is detected using polymerase chain reaction.

43. A kit comprising an acidic medium, a packaging material and instructions for using the acidic medium for enriching a sample with at least one target microbe.

44. The kit of claim 43, further comprising a growth medium.

45. The kit of claim 43, further comprising a pH modifier.

46. The kit of claim 43, further comprising a means to detect the target microbe.

47. The kit of claim 43, wherein the target microbe is a bacterium.

48. The kit of claim 47, wherein the bacterium is enterohemorrhagic Escherichia coli, enteropathogenic Escherichia coli, or enterotoxigenic Escherichia coli.

49. The kit of claim 47, wherein the bacterium is Escherichia coli O157:H7.

50. The kit of claim 47, wherein the bacterium is Shigella.

51. A kit comprising packaging material, an acidic medium, a growth medium, and a means for detecting bacteria.

52. The kit of claim 51, wherein the acidic medium and the growth medium are in liquid form.

53. The kit of claim 51, wherein the acidic medium and the growth medium are in dry form.

54. The kit of claim 53, further comprising sterile water.

55. The kit of claim 51, further comprising a pH modifier.

56. A kit comprising packaging material, culture media, a first pH modifier, and a second pH modifier, wherein addition of the first pH modifier to the culture media produces an acidic medium and addition of the second pH modifier to the acidic medium produces a growth medium.

57. The kit of claim 56, wherein the first pH modifier is an organic acid.

58. The kit of claim 56, wherein the organic acid is selected from the group consisting of lactic acid, formic acid, acetic, proprionic, and butyric.

59. The kit of claim 56, wherein the first pH modifier is an inorganic acid.

60. The kit of claim 56, wherein the first pH modifier is HC1, HF, HBr, H₂SO₄, or H₃PO₄.

61. The kit of claim 56, wherein the second pH modifier is NaOH or KOH.
